(19) United States
(12) Patent Application Publication
(10) Pub. No.: US 2011/0118776 A1
(43) Pub. Date: May 19, 2011

(54) DELIVERY WIRE ASSEMBLY FOR OCCLUSIVE DEVICE DELIVERY SYSTEM

(75) Inventors: Hancun Chen, San Ramon, CA (US); Richard Murphy, Sunnyvale, CA (US); Shana Castelli, San Francisco, CA (US)

(73) Assignee: BOSTON SCIENTIFIC SCIMED, INC., Maple Grove, MN (US)

(21) Appl. No.: 12/945,210

(22) Filed: Nov. 12, 2010

Related U.S. Application Data
(60) Provisional application No. 61/262,394, filed on Nov. 18, 2009.

(51) Int. Cl. A61F 2/01

(52) U.S. Cl. 606/200

(57) ABSTRACT

A delivery wire assembly for delivery of an occlusive device to a location in a patient's vasculature, the assembly having a distal end portion comprising a proximal rigid segment, a distal rigid segment, and a flexible segment disposed between the proximal and distal rigid segments. The assembly also has a delivery wire conduit having a proximal tubular portion coupled to a distal coil portion, the respective tubular and coil portions defining a conduit lumen. A plug is seated in the conduit lumen and secured to an interior surface of the delivery wire conduit. A core wire is disposed in the conduit lumen and having a distal end extending through the plug, so that the plug secures the core wire to the delivery wire conduit. The plug may form a substantially fluid tight seal of the conduit lumen.

### RELATED APPLICATION DATA

The present application claims the benefit under 35 U.S.C. §119 to U.S. provisional patent application Ser. No. 61/262,394, filed Nov. 18, 2009. The foregoing application is hereby incorporated by reference into the present application in its entirety.

### FIELD

The field of the disclosed inventions generally relates to delivery devices for implanting vaso-occlusive devices for establishing an embolus or vascular occlusion in a vessel of a human or veterinary patient.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., describes a vaso-occlusive coil that assumes a linear, helical primary shape when stretched for placement through the lumen of a delivery catheter, and a folded, convoluted secondary shape when released from the delivery catheter and deposited in the vasculature.

In order to deliver the vaso-occlusive coils to a desired site in the vasculature, e.g., within an aneurismal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive coil(s) into the aneurysm once the guidewire is withdrawn. A delivery or “pusher” wire is then passed through the micro-catheter, until a vaso-occlusive coil coupled to a distal end of the delivery wire is extended out of the distal end opening of the micro-catheter and into the aneurysm. The vaso-occlusive device is then released or “detached” from the end delivery wire, and the delivery wire is withdrawn back through the catheter. Depending on the particular needs of the patient, one or more additional occlusive devices may be pushed through the catheter and released at the same site.

One well-known way to release a vaso-occlusive coil from the end of the delivery wire is through the use of an electrolytically severable junction, which is a small exposed section or detachment zone located along a distal end portion of the delivery wire. The detachment zone is typically made of stainless steel and is located just proximal of the vaso-occlusive device. An electrolytically severable junction is susceptible to electrolysis and disintegrates when the delivery wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive delivery wire completes a circuit with a return electrode, and the detachment zone disintegrates due to electrolysis.

In a “monopolar” system, a return electrode (i.e., cathode) is attached to the patient's skin and/or a conductive needle is inserted through the skin at a remote site. In a “bipolar” system, the return electrode is located on the delivery wire, electrically insulated from the conductive path ending at the detachment zone (i.e., anode). The anode is typically made of an insulated core wire that runs through the delivery wire and is attached to the electrical contact at the proximal end. An exposed portion of the core wire forms the detachment zone at the distal end. The anode electrical contact may be a metallic tube secured to the proximal end of the delivery wire. Whether monopolar or bipolar, the distal end of the delivery wire can have a myriad of features, including a coil forming the delivery wire conduit, a reflowed polymer (e.g., polyether block amide or Pebax®) section covering the conduit, and an adhesive filled stopper coil plug inserted into the distal end of the conduit. These features result in the formation a relatively long stiff zone at the distal end of the delivery wire with sub-zones of extra stiffness when the features overlap. Perceived problems with current vaso-occlusive coil delivery systems include the long stiff zone at the distal end of the delivery wire, which reduces flexibility as the delivery wire is pushed through the micro-catheter. Reduced flexibility, combined with the orthogonal and axial forces exerted on the delivery wire as it is pushed through the micro-catheter, results in reduced durability.

### SUMMARY

Embodiments of the disclosed inventions are directed to delivery wire assemblies for delivery of occlusive devices to a location in a patient's vasculature.

In one embodiment, the delivery wire assembly has a distal end portion comprising a proximal rigid segment, a distal rigid segment, and a flexible segment disposed between the proximal and distal rigid segments. The delivery wire assembly may further include a delivery wire conduit having a proximal tubular portion coupled to a distal coil portion, the respective tubular and coil portions defining a conduit lumen. A plug may be seated in the conduit lumen and secured to an interior surface of the delivery wire conduit. A core wire may be disposed in the conduit lumen and having a distal end extending through the plug, so that the plug secures the core wire to the delivery wire conduit. The plug may form a substantially fluid tight seal of the conduit lumen. In one such embodiment, the plug comprises a stopper coil and an adhesive wicked into the stopper coil. In one such embodiment, the proximal rigid segment comprises a proximal region of the distal coil portion and a reflowed polymer section covering the proximal region, the flexible segment comprises an unsheathed middle region of the distal coil portion, and the distal rigid segment comprises the plug and a distal region of the distal coil portion covering the plug.

In another embodiment, the delivery wire assembly has a distal end portion comprising first, second and third rigid segments, along with first and second flexible segments, wherein the first flexible segment is disposed between the first and second rigid segments, and the second flexible segment is disposed between the second and third rigid segments. Such embodiment may further include a delivery wire conduit having a proximal tubular portion coupled to a distal coil portion, the respective tubular and coil portions defining a conduit lumen. A plug may be seated in the conduit lumen and secured to an interior surface of the delivery wire conduit. A core wire may be disposed in the conduit lumen and having a distal end extending through the plug, so that the plug secures the core wire to the delivery wire conduit. The plug may form a substantially fluid tight seal of the conduit lumen.

In one such embodiment, the plug includes a stopper coil and an adhesive. The stopper coil has a proximal open pitch area, a closed pitch area disposed distal to the proximal open pitch area, and a distal open pitch area disposed distal to the closed pitch area, the adhesive wicked into the proximal and distal open pitch areas, the closed pitch area disposed between the proximal and distal open pitch areas. The plug may also comprise a thin polymer tube disposed around the part of the core wire covered by the closed pitch area of the stopper coil. In such embodiments, the first rigid segment may comprise a proximal region of the distal coil portion and a reflowed polymer section covering the proximal region, and the first flexible segment may comprise an unsheathed mid-proximal region of the distal coil portion. In some such embodiments, the second rigid segment comprises the proximal open pitch area of the plug and a middle region of the distal coil portion covering the proximal open pitch area of the plug, the second flexible segment comprises the closed pitch area of the plug and a mid-distal region of the distal coil portion covering the closed pitch area of the plug, and the third rigid segment comprises the distal open pitch area of the plug and an unsheathed distal region of the distal coil portion covering the plug.

In still another embodiment, a delivery wire assembly for delivery of an occlusive device to a location in a patient's vasculature includes an assembly proximal end and an assembly distal end, the assembly distal end comprising a proximal rigid segment, a proximal flexible segment disposed distal to the proximal rigid segment, a distal rigid segment disposed distal to the proximal flexible segment, and a distal flexible segment disposed distal to the distal rigid segment, wherein the proximal flexible segment is disposed between the proximal and distal rigid segments. The delivery wire assembly further comprises a delivery wire conduit having a proximal tubular portion coupled to a distal coil portion, the respective tubular and coil portions defining a conduit lumen. A plug is seated in the conduit lumen and secured to an interior surface of the delivery wire conduit. A core wire is disposed in the conduit lumen and having a distal end extending through the plug, so that the plug secures the core wire to the delivery wire conduit. The plug may form a substantially fluid tight seal of the conduit lumen.

In one such embodiment, the plug comprises a stopper coil having an open pitch area and a closed pitch area disposed distal to the open pitch area, wherein an adhesive is wicked into the open pitch area. The proximal rigid segment comprises a proximal region of the distal coil portion and a reflowed polymer section covering the proximal region. The proximal flexible segment comprises an unsheathed mid-proximal region of the distal coil portion. The distal rigid segment comprises the open pitch area of the plug and a mid-distal region of the distal coil portion covering the open pitch area of the plug. The distal flexible segment comprises the closed pitch area of the plug and a distal region of the distal coil portion covering the closed pitch area of the plug.

Such embodiments may optionally further comprise a middle flexible segment disposed between the proximal flexible segment and the distal rigid segment, wherein the proximal flexible segment is disposed between the proximal rigid segment and the middle flexible segment. Again, the plug comprises a stopper coil and an adhesive, the stopper coil comprising a proximal closed pitch area, an open pitch area disposed distal to the proximal closed pitch area, and a distal closed pitch area disposed distal to the open pitch area, the adhesive wicked into the open pitch area, and the open pitch area disposed between the proximal and distal closed pitch areas.

In one such embodiment, the proximal rigid segment comprises a proximal region of the distal coil portion and a reflowed polymer section covering the proximal region, the proximal flexible segment comprises an unsheathed mid-proximal region of the distal coil portion, the middle flexible segment comprises the proximal closed pitch area of the plug and a middle region of the distal coil portion covering the proximal closed pitch area of the plug, the distal rigid segment comprises the open pitch area of the plug and a mid-distal region of the distal coil portion covering the open pitch area of the plug, and the distal flexible segment comprises the distal closed pitch area of the plug and a distal region of the distal coil portion covering the distal closed pitch area of the plug.

Other and further aspects and features of the disclosed inventions will be evident from reading the following detailed description of the preferred embodiments, which are intended to illustrate, not limit, the disclosed inventions.

### BRIEF DESCRIPTION OF THE DRAWINGS

Referring now to the drawings in which like reference numbers represent corresponding parts throughout, and in which:

FIG. 1 illustrates an occlusive coil delivery system, according to one embodiment.

FIG. 2 illustrates an occlusive coil in a natural state mode, illustrating one exemplary secondary configuration.

FIGS. 3 and 4 are detailed longitudinal cross-sectional views of a delivery wire assembly according to one embodiment. FIG. 3 includes labels for regions of the distal coil portion of the delivery wire conduit. FIG. 4 includes labels for segments of the delivery wire assembly.

FIGS. 5-7 are detailed longitudinal cross-sectional views of a delivery wire assembly according to another embodiment. FIGS. 5 and 7 include labels for regions of the distal coil portion of the delivery wire conduit. FIG. 6 includes labels for segments of the delivery wire assembly.

FIGS. 8 and 9 are detailed longitudinal cross-sectional views of a delivery wire assembly according to one embodiment. FIG. 8 includes labels for regions of the distal coil portion of the delivery wire conduit. FIG. 9 includes labels for segments of the delivery wire assembly.

FIGS. 10 and 11 are detailed longitudinal cross-sectional views of a delivery wire assembly according to one embodiment, wherein FIG. 10 includes labels for regions of the distal coil portion of the delivery wire conduit, and FIG. 11 includes labels for segments of the delivery wire assembly.

FIGS. 12, 13, 14, and 15 are tables summarizing the components of various segments of the delivery wire assemblies depicted in FIGS. 3 and 4; 5-7; 8 and 9; and 10 and 11, respectively.

### DETAILED DESCRIPTION OF THE ILLUSTRATED EMBODIMENTS

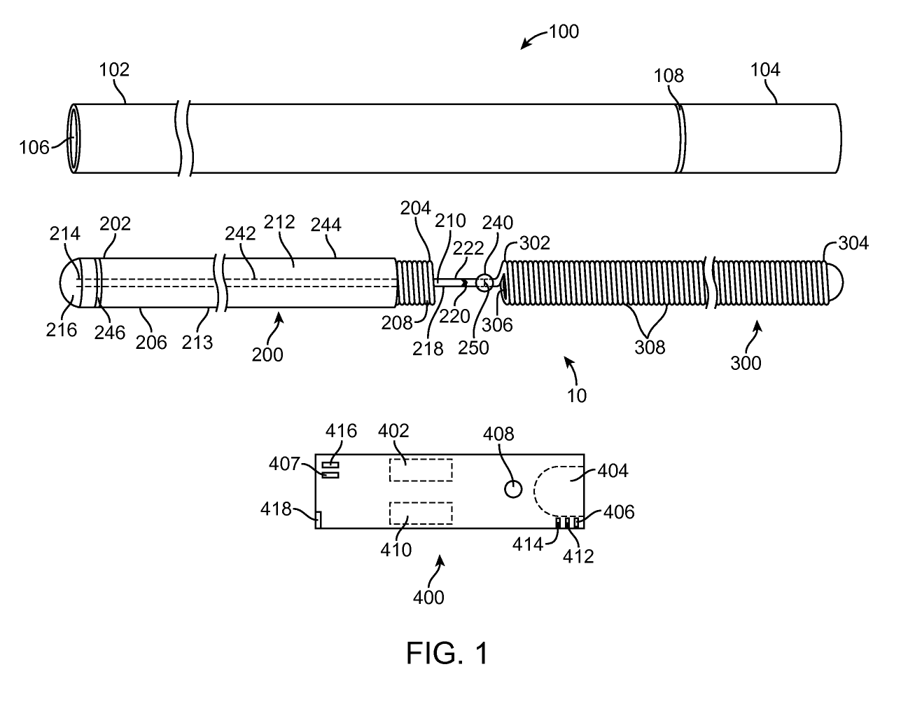

FIG. 1 illustrates an occlusive coil delivery system 10 according to one embodiment of the disclosed inventions. The system 10 includes a number of subcomponents or sub-systems, including a delivery catheter 100, a delivery wire assembly 200, an occlusive coil 300, and a power supply 400. The delivery catheter 100 includes a proximal end 102, a distal end 104, and a lumen 106 extending between the proximal and distal ends 102, 104. The lumen 106 of the delivery catheter 100 is sized to accommodate axial movement of the delivery wire assembly 200. Further, the lumen 106 is sized for the passage of a guidewire (not shown), which may optionally be used to properly guide the delivery catheter 100 to the appropriate delivery site. The delivery catheter 100 may include a braided-shaft construction of stainless steel flat wire that is encapsulated or surrounded by a polymer coating. By way of non-limiting example, HYDROLENE® is a polymer coating that may be used to cover the exterior portion of the delivery catheter 100. Of course, the system 10 is not limited to a particular construction or type of delivery catheter 100 and other constructions known to those skilled in the art may be used for the delivery catheter 100.

The inner lumen 106 may be advantageously coated with a lubricious coating such as PTFE to reduce frictional forces between the delivery catheter 100 and the respective delivery wire assembly 200 and occlusive coil 300 being moved axially within the lumen 106. The delivery catheter 100 may include one or more optional marker bands 108 formed from a radiopaque material that can be used to identify the location of the delivery catheter 100 within the patient's vasculature system using imaging technology (e.g., fluoroscope imaging). The length of the delivery catheter 100 may vary depending on the particular application, but generally is around 150 cm in length. Of course, other lengths of the delivery catheter 100 may be used with the system 10 described herein.

The delivery catheter 100 may include a distal end 104 that is straight as illustrated in FIG. 1. Alternatively, the distal end 104 may be pre-shaped into a specific geometry or orientation. For example, the distal end 104 may be shaped into a “C” shape, an “S” shape, a “J” shape, a 45° bend, a 90° bend. The size of the lumen 106 may vary depending on the size of the respective delivery wire assembly 200 and occlusive coil 300, but generally the diameter lumen 106 of the delivery catheter 100 (I.D. of delivery catheter 100) is less than about 0.02 inches. The delivery catheter 100 is known to those skilled in the art as a microcatheter. While not shown in FIG. 1, the delivery catheter 100 may be utilized with a separate catheter that aids in guiding the delivery catheter 100 to the appropriate location within the patient's vasculature.

Still referring to FIG. 1, the system 10 includes a delivery wire assembly 200 configured for axial movement within the lumen 106 of the delivery catheter 100. The delivery wire assembly 200 generally includes a proximal end 202 and a distal end 204. The delivery wire assembly 200 includes a delivery wire conduit 213, which has a proximal tubular portion 206 and a distal coil portion 208. The proximal tubular portion 206 may be formed from, for example, a flexible stainless steel hypotube. The distal coil portion 208 may be formed from, for example, stainless steel wire. The distal coil portion 208 may be joined to the proximal tubular portion 206 in an end-to-end arrangement. The delivery wire conduit 213 is configured to carry the load of the compressive forces as the delivery wire assembly 200 is pushed distally through the delivery catheter 100.

The delivery wire assembly 200 further includes a core wire 210 that extends from the proximal end 202 of the delivery wire assembly 200 to a location that is distal with respect to the distal end 204 of the delivery wire assembly 200. The core wire 210 is disposed within a conduit lumen 212 that extends within an interior portion of the delivery wire conduit 213. The core wire 210 is formed from an electrically conductive material such as stainless steel wire. The proximal end 214 of the core wire 210 (shown in phantom) is electrically coupled to an electrical contact 216 located at the proximal end 202 of the delivery wire assembly 200. The electrical contact 216 is configured to interface with a corresponding electrical contact (not shown) in the power supply 400. The core wire 210 is connected to the delivery wire conduit 213 as described below. The core wire 210 functions as a tether to the occlusive coil 300, such that when the delivery wire assembly 200 is pulled proximally, the occlusive coil 300 can also be withdrawn prior to coil detachment.

A portion of the core wire 210 is advantageously coated with an insulative coating 218. The insulative coating 218 may include polyimide. The entire length of the core wire 210 is coated with an insulative coating 218, except for the proximal end 214 of the core wire 210 that contacts the electrical contact 216, and a small region 220 located in a portion of the core wire 210 that extends distally with respect to the distal end 204 of the delivery wire assembly 200. This latter, “bare” portion of the core wire 210 forms the electrolytic detachment zone 220, which dissolves upon application of electrical current from the power supply 400.

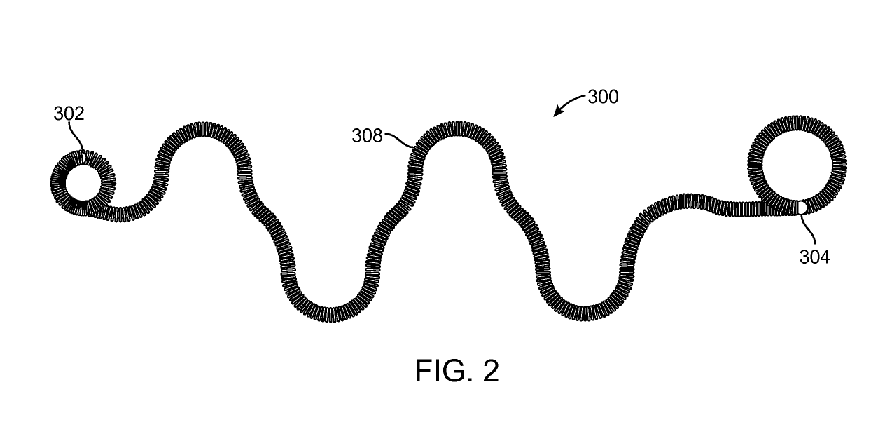

Still referring to FIG. 1, the occlusive coil 300 includes a proximal end 302, a distal end 304, and a lumen 306 extending there between. The occlusive coil 300 is generally made from a biocompatible metal such as platinum or a platinum alloy (e.g., platinum-tungsten alloy). The occlusive coil 300 generally includes a straight configuration (as illustrated in FIG. 1) when the occlusive coil 300 is loaded within the delivery catheter 100. Upon release, the occlusive coil 300 generally takes a secondary shape which may include three-dimensional helical configurations such as those illustrated in FIG. 2. The occlusive coil 300 includes a plurality of coil windings 308. The coil windings 308 are generally helical about a central axis disposed along the lumen 306 of the occlusive coil 300. The occlusive coil 300 may have a closed pitch configuration as illustrated in FIG. 1. Of course, the system 10 described herein may be used with occlusive coils 300 or other structures having a variety of configurations, and is not limited to occlusive coils 300 having certain sizes or configurations. Additional features or components might be used to provide mechanical interlock between the delivery wire 200 and occlusive coil 300.

The distal end 222 of the core wire 210 is connected to the proximal end 302 of the occlusive coil 300 at a junction 250. Various techniques and devices can be used to connect the core wire 210 to the occlusive coil 300, including laser melting, and laser tack, spot, and continuous welding. It is preferable to apply a non-conductive adhesive 240 to cover the junction 250 formed between the distal end 222 of the core wire 210 and the proximal end 302 of the occlusion coil 300. The non-conductive adhesive 240 may include an epoxy material which is cured or hardened through the application of heat or UV radiation. For example, the non-conductive adhesive 240 may include a thermally cured, two-part epoxy such as EPO-TEK® 353ND-4 available from Epoxy Technology, Inc., 14 Fortune Drive, Billerica, Mass. The non-conductive adhesive 240 encapsulates the junction 250 and increases its mechanical stability.

Still referring to FIG. 1, the system 10 further includes a power supply 400 for supplying direct current to the core wire 210, which contains the electrolytic detachment zone 220. In the presence of an electrically conductive fluid (including a physiological fluid such as blood, or an electrically conductive flushing solution such as saline), activation of the power supply 400 causes electrical current to flow in a circuit including the electrical contact 216, the core wire 210, the electrolytic detachment zone 220, and a return electrode contact 246. After several seconds (generally less than about 10 seconds), the sacrificial electrolytic detachment zone 220 dissolves, and the occlusive coil 300 separates form the core wire 210.

The power supply 400 preferably includes an onboard energy source, such as batteries (e.g., a pair of AAA batteries), along with drive circuitry 402. The drive circuitry 402 may include one or more microcontrollers or processors configured to output a driving current. The power supply 400 illustrated in FIG. 1 includes a receptacle 404 configured to receive and mate with the proximal end 202 of the delivery wire assembly 200. Upon insertion of the proximal end 202 into the receptacle 404, the electrical contacts 216, 246 disposed on the delivery wire assembly 200 electrically couple with corresponding contacts (not shown) located in the power supply 400.

A visual indicator 406 (e.g., LED light) is used to indicate when the proximal end 202 of delivery wire assembly 200 has been properly inserted into the power supply 400. Another visual indicator 407 is activated if the onboard energy source needs to be recharged or replaced. The power supply 400 includes an activation trigger or button 408 that is depressed by the user to apply the electrical current to the sacrificial electrolytic detachment zone 220. Once the activation trigger 408 has been activated, the driver circuitry 402 automatically supplies current until detachment occurs. The drive circuitry 402 typically operates by applying a substantially constant current, e.g., around 1.5 mA. The power supply 400 may include optional detection circuitry 410 that is configured to detect when the occlusive coil 300 has detached from the core wire 210. The detection circuitry 410 may identify detachment based upon a measured impedance value. A visual indicator 412 may indicate when the power supply 400 is supplying adequate current to the sacrificial electrolytic detachment zone 220. Another visual indicator 414 may indicate when the occlusive coil 300 has detached from the core wire 210. As an alternative to the visual indicator 414, an audible signal (e.g., beep) or even tactile signal (e.g., vibration or buzzer) may be triggered upon detachment. The detection circuitry 410 may be configured to disable the drive circuitry 402 upon sensing detachment of the occlusive coil 300.

The power supply 400 may also contain another visual indicator 416 that indicates to the operator when non-bipolar delivery wire assembly is inserted into the power supply 400. As explained in the background above, non-bipolar delivery wire assemblies use a separate return electrode that typically is in the form of a needle that was inserted into the groin area of the patient. The power supply 400 is configured to detect when a non-bipolar delivery wire assembly has been inserted. Under such situations, the visual indicator 416 (e.g., LED) is turned on and the user is advised to insert the separate return electrode (not shown in FIG. 1) into a port 418 located on the power supply 400.

Still referring to FIG. 1, the core wire 210 forms a first conductive path 242 between the electrical contact 216 and the electrolytic detachment zone 220. This first conductive path 242 comprises the anode (+) of the electrolytic circuit when the delivery wire assembly 200 is operatively coupled to the power supply 400. A second conductive path 244, the return path, is formed by the proximal tubular portion 206 and a distal coil portion 208 of the delivery wire conduit 213. The second conductive path 244 is electrically isolated from the first conductive path 242. The second conductive path 244 comprises the cathode (−) or ground electrode for the electrical circuit.

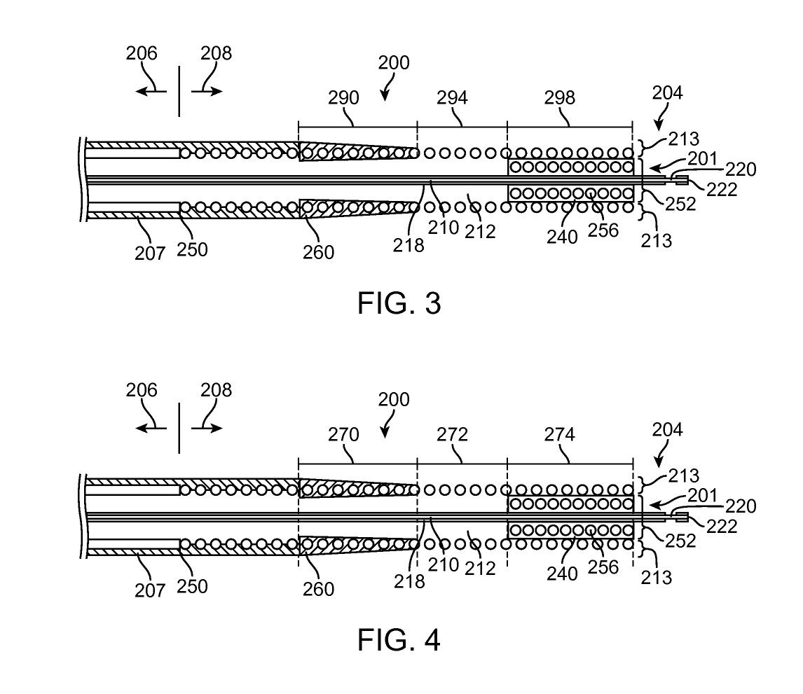

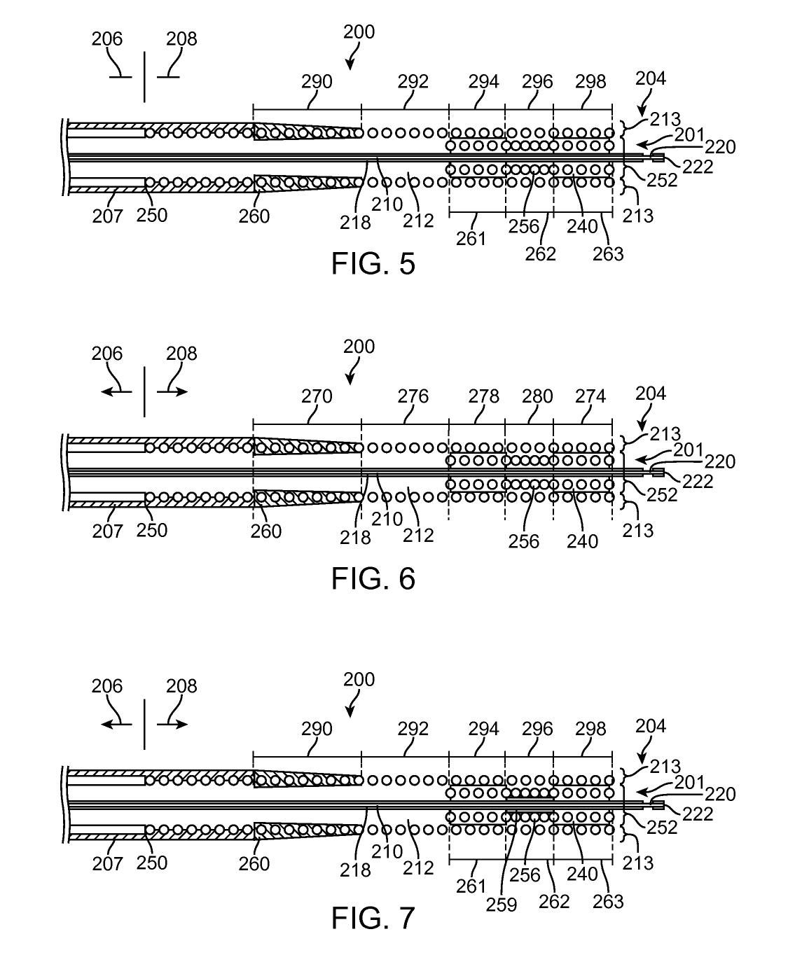

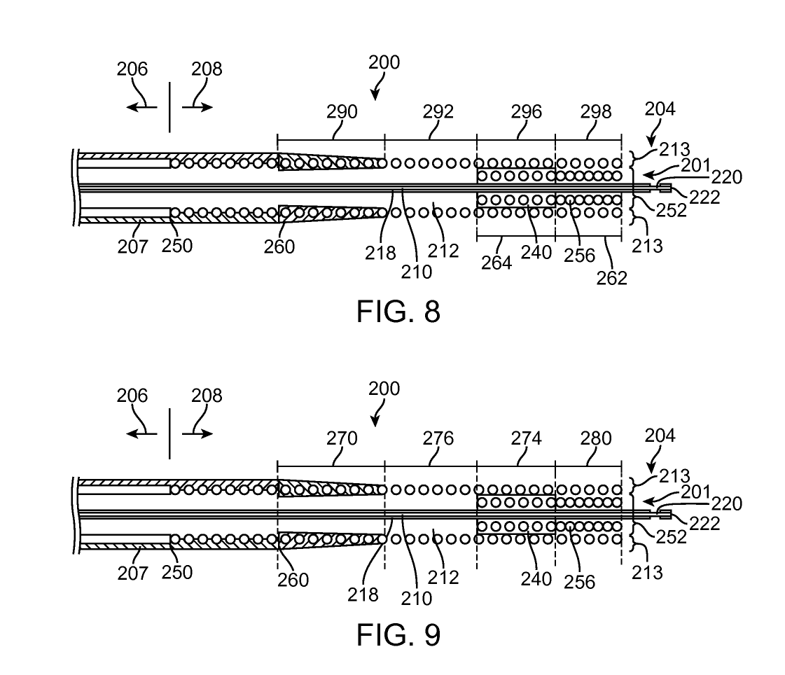

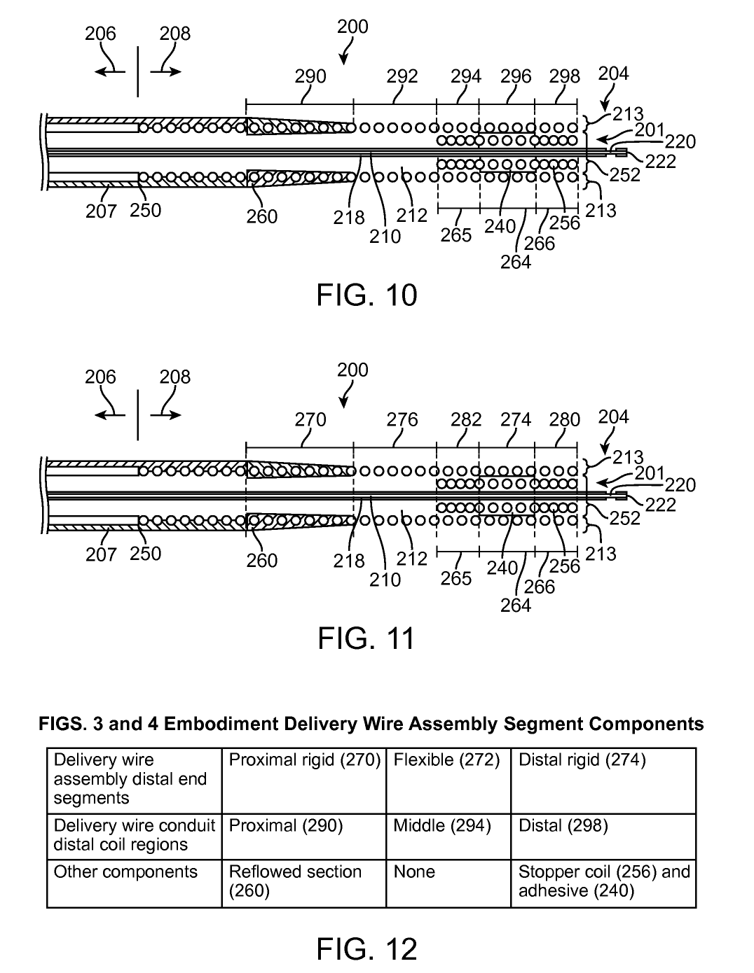

A ground contact 246 for the second conductive path 244 may be disposed on a proximal end of the tubular portion 206 of the delivery wire conduit 213. In one embodiment, the ground contact 246 is simply an exposed portion of the tubular portion 206 since the tubular portion 206 is part of the second conductive path 244. For instance, a proximal portion of the tubular portion 206 that is adjacent to the electrical contact 216 may be covered with an insulative laminated polymer sheath 207, such as laminated Pebax®, as illustrated in FIG. 3. An exposed region of the tubular portion 206 that does not have the insulative coating may form the ground contact 246. Alternatively, the ground contact 246 may be a ring type electrode or other contact that is formed on the exterior of the tubular portion 206.

The ground contact 246 is configured to interface with a corresponding electrical contact (not shown) in the power supply 400 when the proximal end 202 of the delivery wire assembly 200 is inserted into the power supply 400. The ground contact 246 of the second conductive path 244 is, of course, electrically isolated with respect to the electrical contact 216 of the first conductive path 242.

FIG. 2 illustrates one exemplary configuration of an occlusive coil 300 in a natural state. In the natural state, the occlusive coil 300 transforms from the straight configuration illustrated in, for instance, FIG. 1 into a secondary shape. The secondary shaped may include both two and three dimensional shapes of a wide variety. FIG. 2 is just one example of a secondary shape of an occlusive coil 300 and other shapes and configurations are contemplated to fall within the scope of the disclosed embodiments. Also, the occlusive coil 300 may incorporate synthetic fibers over all or a portion of the occlusive coil 300 as is known in the art. These fibers may be attached directly to coil windings 308 or the fibers may be applied to the occlusive coil 300 using a weave or braided configuration.

FIG. 3 illustrates a detailed longitudinal cross-sectional view of the delivery wire assembly 200 according to one embodiment. Similar elements of this embodiment are identified with the same reference numbers as discussed above with respect to FIG. 1. The delivery wire assembly 200 includes a proximal end 202 and a distal end 204 and measures between around 184 cm to around 186 cm in length. The delivery wire assembly 200 includes a delivery wire conduit 213 with a proximal tubular portion 206 and a distal coil portion 208. The proximal tubular portion 206 may be formed from stainless steel hypotube having an outer diameter (OD) of around 0.013 inches and inner diameter (ID) of around 0.005 inches. The length of the hypotube section may be between around 140 cm to around 150 cm.

As seen in FIG. 3, a distal coil portion 208 is joined in end-to-end fashion to the distal face of the proximal tubular portion 206. The joining may be accomplished using a weld or other bond. The distal coil portion 208 may have a length of around 39 cm to around 41 cm in length. The distal coil portion 208 may comprise a coil of around 0.0025 inches×around 0.006 inches. The first dimension generally refers to the OD of the coil wire that forms the coil. The latter dimension generally refers to the internal mandrel used to wind the coil wire around to form the plurality of coil winds and is the nominal ID of the coil. One or more marker coils (not shown) of the distal coil portion 208 may be formed from a radiopaque material. For example, the distal coil portion 208 may include a segment of stainless steel coil (e.g., around 3 cm in length), followed by a segment of platinum coil (which is radiopaque and also around 3 mm in length), followed by a segment of stainless steel coil (e.g., around 37 cm in length), and so on and so forth.

The core wire 210, which runs through the delivery wire conduit 213, terminates at electrical contact 216 at one end and extends distally with respect to the distal coil portion 208 of the delivery wire conduit 213. The core wire 210 is coated with an insulative coating 218 such as polyimide except at the electrolytic detachment zone 220 and the proximal segment coupled to the electrical contact 216. The electrolytic detachment zone 220 is located less than half a millimeter (e.g., about 0.02 mm to about 0.2 mm) distally with respect to the distal end of the distal coil portion 208. The core wire 210 may have an OD of around 0.00175 inches.

As shown in FIG. 3, a plug 252 sits in the distal opening 201 of the delivery wire conduit 213. The plug 252 connects the core wire 210 and the delivery wire conduit 213 at the distal end 204 of the delivery wire assembly 200. The plug 252 transfers sufficient axial force to allow the delivery wire conduit 213 to pull the core wire 210 distally into the catheter 100 and to allow the core wire 210 to pull the delivery wire conduit 213 proximally out of the catheter 100. The plug can also form a substantially fluid tight seal of the distal opening 201 of the delivery wire conduit 213. The plug 252 includes a stopper coil 256, which is held in place by non-conductive adhesive 240 connecting it to the core wire 210 and the inside of the delivery wire conduit 213. The adhesive 240 may include EPO-TEK® 353ND-4 described in more detail above. The stopper coil 256 is made of stainless steel wire. In other embodiments, the stopper coil 256 is also held in place by conductive adhesive, i.e., silver epoxy, connecting it to the inside of the delivery wire conduit 213. The conductive adhesive electrically connects the stopper coil 256 to the ground electrode 246 via the delivery wire conduit 213, making it part of the second conductive path 244.

In the embodiment depicted in FIGS. 3 and 4, a laminated polymer sheath 207 surrounds a portion of the proximal tubular portion 206 and the proximal tubular portion 206 adjacent part of the distal coil portion 208 of the delivery wire conduit 213. The laminated polymer sheath 207 covers the junction 250 formed between the proximal tubular portion 206 and the distal coil portion 208. The laminated polymer sheath 207 may have a length of around 50 cm to around 54 cm. The approximately uniform OD of the laminated polymer sheath 207 may be less than about 0.02 inches and advantageously from about 0.010 to about 0.018 inches. The section of the laminated polymer sheath 207 covering a proximal region 290 of the distal coil portion 208 of the delivery wire conduit 213 is a reflowed section 260 of the polymer sheath 207. The reflowed section 260 may have a length less than about 0.039 inches and preferably from about 0.020 to about 0.035 inches. The OD of the reflowed section 260 may be less than around 0.02 inches and preferably less than around 0.015 inches.

The laminated polymer sheath 207 may be formed from a polyether block amide plastic material (e.g., Pebax® 7233 lamination). The laminated polymer sheath 207 is formed from a polymer tube that is heat laminated to the proximal tubular portion 206 adjacent part of the distal coil portion 208 of the delivery wire conduit 213. During heat lamination, heat is applied to a piece of balloon tubing overlying the polymer tube. In response to this heat treatment, the balloon tubing shrinks, transforming the polymer tube into the laminated polymer sheath 207. After lamination, the balloon tubing is peeled away from the laminated polymer sheath 207 and discarded. Then the polymer at the distal end (about 0.045 to about 0.065 inches from the distal tip) of the distal coil portion 208 is removed to expose the metallic surface of the distal coil portion 208 to form a return cathode. A reflowing process is then applied to distal end of the remaining polymer to form a smooth transition between the laminated and the non-laminated areas of the distal coil portion 208.

The reflowed section 260 may be formed from the polymer tube that was laminated around the proximal region 290 of the distal coil portion 208 of the delivery wire conduit 213. During reflowing, the polymer tube laminated around the proximal region 290 is reheated. The heat and dwell time of the heat are increased for reflowing with use of a hot air nozzle to taper and feather the reflowed section 260 distally. As shown in FIG. 4, the proximal region 290 of the distal coil portion 208 of the delivery wire conduit 213 and the reflowed section 260 form a proximal rigid delivery wire assembly segment 270.

Distal of the proximal region 290 of the distal coil portion 208 of the delivery wire conduit 213 is a middle region 294 of the distal coil portion 208 of the delivery wire conduit 213. The middle region 294 is an area of unsheathed coils from the distal coil portion 208 of the delivery wire conduit 213. The middle region 294 may have a length of around 0.010 inches. The unsheathed middle region 294 forms a flexible delivery wire assembly segment 272, as shown in FIG. 4.

Distal of the middle region 294 of the distal coil portion 208 of the delivery wire conduit 213 is a distal region 298 of the distal coil portion 208 of the delivery wire conduit 213 that covers the plug 252. The distal region 298 may have a length less than about 0.039 inches and advantageously from about 0.020 to about 0.035 inches. The distal region 298 of the distal coil portion 208 of the delivery wire conduit 213, and the stopper coil 256 and adhesive 240 of the plug 256 form a distal rigid delivery wire assembly segment 274, as shown in FIG. 4.

As shown in FIG. 3, the distal coil portion 208 of the delivery wire conduit 213 has three regions: the proximal region 290; the middle region 294; and the distal region 298. These three regions either alone or in combination with other parts of the delivery wire assembly 200, form three segments of the delivery wire assembly 200: the proximal rigid segment 270; the flexible segment 272; and the distal rigid segment 274, respectively, as summarized in FIG. 12.

Positioning of the flexible segment 272 between the proximal rigid segment 270 and the distal rigid segment 274 allows the two rigid segments 270, 274 to change orientation relative to each other about the articulation space of the flexible segment 272. The elimination of overlap between the reflowed section 260 and the plug 252 eliminates long stiff zones and extra stiff sub-zones. This configuration allows sectional bending as the delivery wire assembly 200 is navigated through the highly tortuous paths of a patient's vasculature.

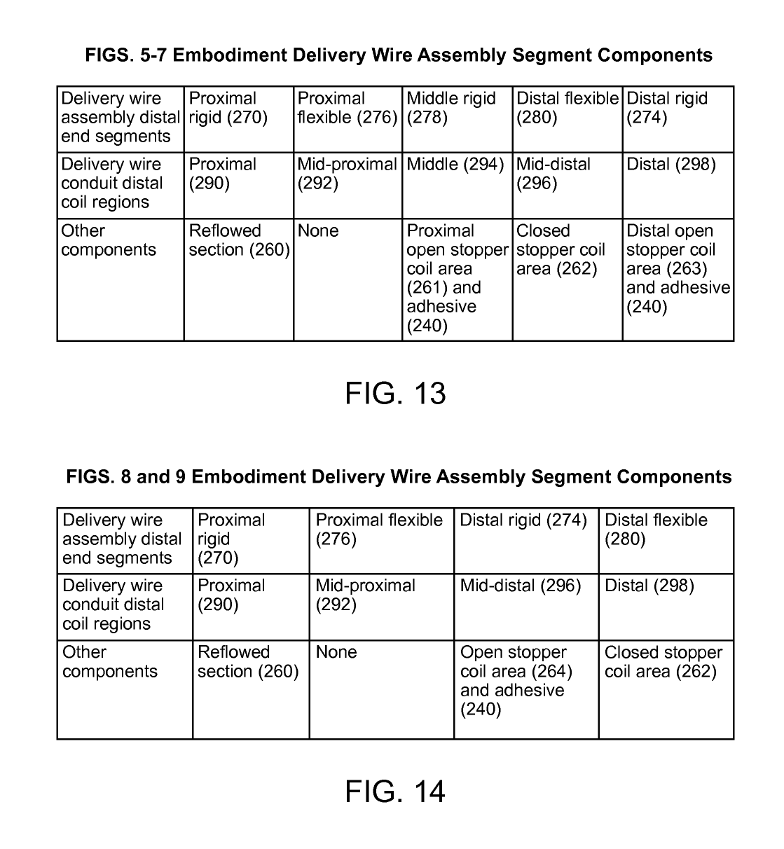

In another embodiment shown in FIGS. 5, 6, and 7, distal of the laminated polymer sheath 207 a proximal region 290 of the distal coil portion 208 of the delivery wire conduit 213 is covered by a reflowed section 260, as described above. As also described above, the proximal region 290 of the distal coil portion 208 of the delivery wire conduit 213 and the reflowed section 260 form a proximal rigid delivery wire assembly segment 270, as shown in FIG. 6. Distal of the proximal region 290 of the distal coil portion 208 of the delivery wire conduit 213 is a mid-proximal region 292 of the distal coil portion 208 of the delivery wire conduit 213. The mid-proximal region 292 is an area of unsheathed coils from the distal coil portion 208 of the delivery wire conduit 213. The mid-proximal region 292 may have a length around 0.010 inches. As shown in FIG. 6, the unsheathed mid-proximal region 292 forms a proximal flexible delivery wire assembly segment 270.

The stopper coil 256 in the plug 252 in this embodiment is divided into three distinct areas: a proximal open pitch area 261; a distal open pitch area 263; and a closed pitch area 262 between the two open pitch areas 261, 263. The pitch of the stopper coil 256 in the open pitch areas 261, 263 is approximately 100%. The pitch of the stopper coil 256 in the closed pitch area 262 is approximately 0%. Consequently, when the adhesive 240 is applied to the stopper coil 256 during construction of the delivery wire assembly 200, the adhesive 240 wicks into the open pitch areas 261, 263, increasing their stiffness. Due to the lack of space between the coils in the closed pitch area 262, the adhesive 240 cannot wick into the closed pitch area 262. The resulting configuration is a flexible closed pitch area 262 between two open pitch areas 261, 263. As shown in FIG. 7, a polymer tube 259 can also be secured around the part of the core wire 210 covered by the closed pitch area 262 to further prevent wicking of adhesive 240 under the closed pitch area 262.

Distal of the mid-proximal region 292 of the distal coil portion 208 of the delivery wire conduit 213 is a middle region 294 of the distal coil portion 208 of the delivery wire conduit 213 that covers the proximal open pitch area 261 of the stopper coil 256. The middle region 294 may have a length less than about 0.039 inches and advantageously from about 0.010 to about 0.015 inches. The middle region 294 of the distal coil portion 208 of the delivery wire conduit 213, the proximal open pitch area 261 of the stopper coil 256, and the adhesive 240 wicked into the proximal open pitch area 261 form a middle rigid delivery wire assembly segment 278 (shown in FIG. 6). Distal of the middle region 294 of the distal coil portion 208 of the delivery wire conduit 213 is a mid-distal region 296 of the distal coil portion 208 of the delivery wire conduit 213 that covers the closed pitch area 262 of the stopper coil 256. The mid-distal region 296 may have a length of around 0.010 inches. The mid-distal region 296 of the distal coil portion 208 of the delivery wire conduit 213 and the closed pitch area 262 of the stopper coil 256 form a distal flexible delivery wire assembly segment 280 (shown in FIG. 6).

Distal of the mid-distal region 296 of the distal coil portion 208 of the delivery wire conduit 213 is a distal region 298 of the distal coil portion 208 of the delivery wire conduit 213 that covers the distal open pitch area 263 of the stopper coil 256. The distal region 298 may have a length less than about 0.039 inches and advantageously from about 0.020 to about 0.035 inches. The distal region 298 of the distal coil portion 208 of the delivery wire conduit 213, the distal open pitch area 263 of the stopper coil 256, and the adhesive 240 wicked into the distal open pitch area 263 form a distal rigid delivery wire assembly segment 274, as shown in FIG. 6.

As shown in FIG. 5, the distal coil portion 208 of the delivery wire conduit 213 has five regions: the proximal region 290; the mid-proximal region 292; the middle region 294; the mid-distal region 296; and the distal region 298. Also, the stopper coil 256 has three areas: the proximal open area 261; the closed area 262; and the distal open area 263. The five regions of the distal coil portion 208 either alone or in combination with other parts of the delivery wire assembly 200, including the three areas of the stopper coil 256, form five segments of the delivery wire assembly 200: the proximal rigid segment 270; the proximal flexible segment 276; the middle rigid segment 278; the distal flexible segment 280; and the distal rigid segment 274, respectively, as summarized in FIG. 13. Increasing the number of rigid and flexible segments in this embodiment increases sectional bending as the delivery wire assembly 200 is navigated through the highly tortuous paths of a patient's vasculature.

In another embodiment, shown in FIGS. 8 and 9, distal of the laminated polymer sheath 207, a proximal region 290 of the distal coil portion 208 of the delivery wire conduit 213 is covered by a reflowed section 260, as described above. Also as described above, the proximal region 290 of the distal coil portion 208 of the delivery wire conduit 213 and the reflowed section 260 form a proximal rigid delivery wire assembly segment 270 (shown in FIG. 9). Distal of the proximal region 290 of the distal coil portion 208 of the delivery wire conduit 213 is a mid-proximal region 292 of the distal coil portion 208 of the delivery wire conduit 213. The mid-proximal region 292 is an area of unsheathed coils from the distal coil portion 208 of the delivery wire conduit 213. The mid-proximal region 292 may have a length of around 0.010 inches. The unsheathed mid-proximal region 292 forms a proximal flexible delivery wire assembly segment 276 (shown in FIG. 9).

The stopper coil 256 in the plug 252 in this embodiment is divided into two distinct areas: an open pitch area 264; and a closed pitch area 262 distal to the open pitch area 264. The pitch of the stopper coil 256 in the open pitch area 264 is approximately 100%. The pitch of the stopper coil 256 in the closed pitch area 262 is approximately 0%. As described above, adhesive 240 will wick into the open pitch area 264, but not the closed pitch area 262, increasing the stiffness of the open pitch area 264. Distal of the mid-proximal region 292 of the distal coil portion 208 of the delivery wire conduit 213 is a mid-distal region 296 of the distal coil portion 208 of the delivery wire conduit 213 that covers the open pitch area 264 of the stopper coil 256. The mid-distal region 296 may have a length less than about 0.039 inches and advantageously from about 0.010 to about 0.020 inches. As shown in FIG. 9, the mid-distal region 296 of the distal coil portion 208 of the delivery wire conduit 213, the open pitch area 264 of the stopper coil 256, and the adhesive 240 wicked into the open pitch area 264 collectively form a distal rigid delivery wire assembly segment 274.

Distal of the mid-distal region 296 of the distal coil portion 208 of the delivery wire conduit 213 is a distal region 298 of the distal coil portion 208 of the delivery wire conduit 213 that covers the closed pitch area 262 of the stopper coil 256. The distal region 298 may have a length of around 0.010 to around 0.020 inches. The distal region 298 of the distal coil portion 208 of the delivery wire conduit 213 and the closed pitch area 262 of the stopper coil 256 form a distal flexible delivery wire assembly segment 280, as shown in FIG. 9.

As shown in FIG. 8, the distal coil portion 208 of the delivery wire conduit 213 has four regions: the proximal region 290; the mid-proximal region 292; the mid-distal region 296; and the distal region 298. Also, the stopper coil 256 has two areas: the open area 264; and the closed area 262. The four regions of the distal coil portion 208 either alone or in combination with other parts of the delivery wire assembly 200, including the two areas of the stopper coil 256, form four segments of the delivery wire assembly 200: the proximal rigid segment 270; the proximal flexible segment 276; the distal rigid segment 274; and the distal flexible segment 280, respectively, as summarized in FIG. 14. Increasing the number of rigid and flexible segments in this embodiment increases sectional bending as the delivery wire assembly 200 is navigated through the highly tortuous paths of a patient's vasculature.

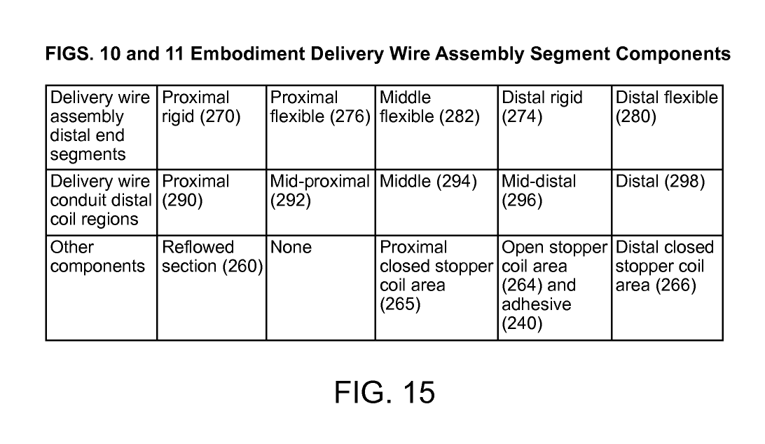

In another embodiment shown in FIGS. 10 and 11, distal of the laminated polymer sheath 207, a proximal region 290 of the distal coil portion 208 of the delivery wire conduit 213 is covered by a reflowed section 260, as described above. Also as described above, The proximal region 290 of the distal coil portion 208 of the delivery wire conduit 213 and the reflowed section 260 form a proximal rigid delivery wire assembly segment 270, as shown in FIG. 11. Distal of the proximal region 290 of the distal coil portion 208 of the delivery wire conduit 213 is a mid-proximal region 292 of the distal coil portion 208 of the delivery wire conduit 213. The mid-proximal region 292 is an area of unsheathed coils from the distal coil portion 208 of the delivery wire conduit 213. The mid-proximal region 292 may have a length of around 0.010 inches. The unsheathed mid-proximal region 292 forms a proximal flexible delivery wire assembly segment 276, as shown in FIG. 11.

The stopper coil 256 in the plug 252 in this embodiment is divided into three distinct areas: a proximal closed pitch area 265; a distal closed pitch area 266; and an open pitch area 264 between the two closed pitch areas 265, 266. The pitch of the stopper coil 256 in the open pitch area 264 is approximately 100%. The pitch of the stopper coil 256 in the closed pitch areas 265, 266 is approximately 0%. As described above, adhesive 240 will wick into the open pitch area 264, but not the closed pitch areas 265, 266, increasing the stiffness of the open pitch area 264.

Distal of the mid-proximal region 292 of the distal coil portion 208 of the delivery wire conduit 213 is a middle region 294 of the distal coil portion 208 of the delivery wire conduit 213 that covers the proximal closed pitch area 265 of the stopper coil 256. The middle region 294 may have a length of around 0.010 inches. The middle region 294 of the distal coil portion 208 of the delivery wire conduit 213 and the proximal closed pitch area 265 of the stopper coil 256 form a middle flexible delivery wire assembly segment 282, as shown in FIG. 11.

Distal of the middle region 294 of the distal coil portion 208 of the delivery wire conduit 213 is a mid-distal region 296 of the distal coil portion 208 of the delivery wire conduit 213 that covers the open pitch area 264 of the stopper coil 256. The mid-distal region 296 may have a length less than about 0.039 inches and advantageously from about 0.010 to about 0.020 inches. The mid-distal region 296 of the distal coil portion 208 of the delivery wire conduit 213, the open pitch area 264 of the stopper coil 256, and the adhesive 240 wicked into the open pitch area 264 form a distal rigid delivery wire assembly segment 274, as shown in FIG. 11.

Distal of the mid-distal region 296 of the distal coil portion 208 of the delivery wire conduit 213 is a distal region 298 of the distal coil portion 208 of the delivery wire conduit 213 that covers the distal closed pitch area 266 of the stopper coil 256. The distal region 298 may have a length of around 0.010 inches. The distal region 298 of the distal coil portion 208 of the delivery wire conduit 213 and the distal closed pitch area 266 of the stopper coil 256 collectively form a distal flexible delivery wire assembly segment 280, as shown in FIG. 11.

As shown in FIG. 10, the distal coil portion 208 of the delivery wire conduit 213 has five regions: the proximal region 290; the mid-proximal region 292; the middle region 294; the mid-distal region 296; and the distal region 298. Also, the stopper coil 256 has three areas: the proximal closed area 265; the open area 264; and the distal closed area 266. The five regions of the distal coil portion 208 either alone or in combination with other parts of the delivery wire assembly 200, including the three areas of the stopper coil 256, form five segments of the delivery wire assembly 200: the proximal rigid segment 270; the proximal flexible segment 276; the middle flexible segment 282; distal rigid segment 274; and the distal flexible segment 280, respectively, as summarized in FIG. 15. Increasing the number of rigid and flexible segments in this embodiment increases sectional bending as the delivery wire assembly 200 is navigated through the highly tortuous paths of a patient's vasculature.

While various embodiments of the disclosed inventions have been shown and described, they are presented for purposes of illustration, and not limitation. Various modifications may be made to the illustrated and described embodiments (e.g., the dimensions of various parts) without departing from the scope of the disclosed inventions, which are limited and defined only by the following claims and their equivalents.

What is claimed is:

1. A delivery wire assembly for delivery of an occlusive device to a location in a patient's vasculature, the assembly having a distal end portion comprising:
a proximal rigid segment;
a distal rigid segment; and
a flexible segment disposed between the proximal and distal rigid segments.

2. The delivery wire assembly of claim 1, further comprising:
a delivery wire conduit having a proximal tubular portion coupled to a distal coil portion, the respective tubular and coil portions defining a conduit lumen;
a plug seated in the conduit lumen and secured to an interior surface of the delivery wire conduit; and
a core wire disposed in the conduit lumen and having a distal end extending through the plug, wherein the plug secures the core wire to the delivery wire conduit.

3. The delivery wire assembly of claim 2, wherein the plug forms a substantially fluid tight seal of the conduit lumen.

4. The delivery wire assembly of claim 2, wherein the plug transfers sufficient axial force to allow the delivery wire conduit to pull the core wire distally into the catheter, and to allow the core wire to pull the delivery wire conduit proximally out of the catheter.

5. The delivery wire assembly of claim 2, the plug comprising a stopper coil and an adhesive wicked into the stopper coil.

6. The delivery wire assembly of claim 2, wherein the proximal rigid segment comprises a proximal region of the distal coil portion and a reflowed polymer section covering the proximal region, the flexible segment comprises an unsheathed middle region of the distal coil portion, and the distal rigid segment comprises the plug and a distal region of the distal coil portion covering the plug.

7. A delivery wire assembly for delivery of an occlusive device to a location in a patient's vasculature, the assembly having a distal end portion comprising:
first, second and third rigid segments; and
first and second flexible segments, wherein the first flexible segment is disposed between the first and second rigid segments, and the second flexible segment is disposed between the second and third rigid segments.

8. The delivery wire assembly of claim 7, further comprising:
a delivery wire conduit having a proximal tubular portion coupled to a distal coil portion, the respective tubular and coil portions defining a conduit lumen;
a plug seated in the conduit lumen and secured to an interior surface of the delivery wire conduit; and
a core wire disposed in the conduit lumen and having a distal end extending through the plug, wherein the plug secures the core wire to the delivery wire conduit.

9. The delivery wire assembly of claim 8, wherein the plug forms a substantially fluid tight seal of the conduit lumen.

10. The delivery wire assembly of claim 8, the plug comprising a stopper coil and an adhesive.

11. The delivery wire assembly of claim 10, wherein the stopper coil comprises a proximal open pitch area, a closed pitch area disposed distal to the proximal open pitch area, and a distal open pitch area disposed distal to the closed pitch area, the adhesive wicked into the proximal and distal open pitch areas, and the closed pitch area disposed between the proximal and distal open pitch areas.

12. The delivery wire assembly of claim 11, the plug further comprising a thin polymer tube disposed around the part of the core wire covered by the closed pitch area of the stopper coil.

13. The delivery wire assembly of claim 8, wherein the first rigid segment comprises a proximal region of the distal coil portion and a reflowed polymer section covering the proximal region, and the first flexible segment comprises an unsheathed mid-proximal region of the distal coil portion.

14. The delivery wire assembly of claim 10, wherein the second rigid segment comprises the proximal open pitch area of the plug and a middle region of the distal coil portion covering the proximal open pitch area of the plug, the second flexible segment comprises the closed pitch area of the plug and a mid-distal region of the distal coil portion covering the closed pitch area of the plug, and the third rigid segment comprises the distal open pitch area of the plug and a distal region of the distal coil portion covering the distal open pitch area of the plug.

15. A delivery wire assembly for delivery of an occlusive device to a location in a patient's vasculature, comprising:
an assembly proximal end;
an assembly distal end, comprising
a proximal rigid segment,
a proximal flexible segment disposed distal to the proximal rigid segment,
a distal rigid segment disposed distal to the proximal flexible segment, and
a distal flexible segment disposed distal to the distal rigid segment,
wherein the proximal flexible segment is disposed between the proximal and distal rigid segments;
a delivery wire conduit having a proximal tubular portion coupled to a distal coil portion, the respective tubular and coil portions defining a conduit lumen;
a plug seated in the conduit lumen and secured to an interior surface of the delivery wire conduit; and
a core wire disposed in the conduit lumen and having a distal end extending through the plug, wherein the plug secures the core wire to the delivery wire conduit.

16. The delivery wire assembly of claim 15, the plug comprising a stopper coil comprising an open pitch area and a closed pitch area disposed distal to the open pitch area, wherein an adhesive is wicked into the open pitch area.

17. The delivery wire assembly of claim 16, wherein
the proximal rigid segment comprises a proximal region of the distal coil portion and a reflowed polymer section covering the proximal region,
the proximal flexible segment comprises an unsheathed mid-proximal region of the distal coil portion,
the distal rigid segment comprises the open pitch area of the plug and a mid-distal region of the distal coil portion covering the open pitch area of the plug, and
the distal flexible segment comprises the closed pitch area of the plug and a distal region of the distal coil portion covering the closed pitch area of the plug.

18. The delivery wire assembly of claim 16, further comprising a middle flexible segment disposed between the proximal flexible segment and the distal rigid segment, wherein the proximal flexible segment is disposed between the proximal rigid segment and the middle flexible segment.

19. The delivery wire assembly of claim 18, the plug comprising a stopper coil and an adhesive, wherein the stopper coil comprises a proximal closed pitch area, an open pitch area disposed distal to the proximal closed pitch area, and a distal closed pitch area disposed distal to the open pitch area, the adhesive wicked into the open pitch area, the open pitch area disposed between the proximal and distal closed pitch areas.

20. The delivery wire assembly of claim 19, wherein
the proximal rigid segment comprises a proximal region of the distal coil portion and a reflowed polymer section covering the proximal region,
the proximal flexible segment comprises an unsheathed mid-proximal region of the distal coil portion,
the middle flexible segment comprises the proximal closed pitch area of the plug and a middle region of the distal coil portion covering the proximal closed pitch area of the plug,
the distal rigid segment comprises the open pitch area of the plug and a mid-distal region of the distal coil portion covering the open pitch area of the plug, and
the distal flexible segment comprises the distal closed pitch area of the plug and a distal region of the distal coil portion covering the distal closed pitch area of the plug.
